(19) United States
(12) Patent Application Publication
(10) Pub. No.: US 2005/0215791 A1
(43) Pub. Date: Sep. 29, 2005

(54) Process for preparing aripiprazole

(76) Inventors: Ben-Zion Dolitzky, Petach Tiqva (IL); Ori Lerman, Givatayim (IL)

Correspondence Address: KENYON & KENYON, ONE BROADWAY, NEW YORK, NY, 10004, US

(21) Appl. No.: 11/053,645

(22) Filed: Feb. 7, 2005

Related U.S. Application Data
(60) Provisional application No. 60/542,412, filed on Feb. 5, 2004.
(60) Provisional application No. 60/645,160, filed on Jan. 18, 2005.

(51) Int. Cl. C07D 43/02

(52) U.S. Cl. 544/363

(57) ABSTRACT

The invention encompasses the synthesis of aripiprazole from BBQ and DCP, and comprises mixing 7-(4-bromobutoxy)-3,4-dihydrocarbostyril (BBQ) and 1-(2,3-dichlorophenyl)piperazine hydrochloride (DCP) in the presence of at least one base and at least one organic solvent to form a reaction mixture; heating the reaction mixture for a sufficient amount of time to effect the reaction; and isolating aripiprazole. The invention also encompasses the use of phase transfer catalysts in synthesizing aripiprazole from BBQ and DCP.

### RELATED US APPLICATION DATA

This application claims the benefit of U.S. provisional application No. [Attorney Docket No. 1662/78203], filed Jan. 18, 2005 and U.S. provisional application No. 60/542,412, filed Feb. 5, 2004.

### FIELD OF THE INVENTION

The invention is directed to processes for preparing aripiprazole using the intermediate BBQ (7-(4-bromobutoxy)-3,4-dihydrocarbostyril) and DCP (1-(2,3-dichlorophenyl)piperazine hydrochloride). The process of preparing aripiprazole may include using phase transfer catalysts.

### BACKGROUND OF THE INVENTION

Schizophrenia is the most common type of psychosis caused by an excessive neurotransmission activity of the dopaminergic nervous system in the central nervous system. A number of drugs have been developed having the activity to block the neurotransmission of dopaminergic receptor in the central nervous system. For example, among the drugs developed are phenothiazine-type compounds such as chlorpromazine; butyrophenone-type compounds such as haloperidol; and benzamide-type compounds such as sulpiride. The drugs are used to improve so-called positive symptoms in the acute period of schizophrenia such as hallucinations, delusions, excitations, and the like.

Many drugs for treating schizophrenia, however, are not effective for improving the so-called negative symptoms which are observed in the chronic period of schizophrenia such as apathy, emotional depression, hypopsychosis, and the like. The drugs currently used have produced undesired side effects such as akathisia, dystonia, Parkinsonism dyskinesia, and late dyskinesia, which are caused by blocking the neurotransmission of dopaminergic receptor in the striate body.

Aripiprazole is a pyschotropic drug that exhibits high affinity for dopamine D₂ and D₃, serotonin 5-HT_{1A} and 5-HT_{2A} receptors, moderate affinity for dopamine D₄, serotonin 5-HT_{2C} and 5-HT₇, α₁-adrenergic and histamine H₁ receptors, and moderate affinity for the serotonin reuptake site. Also, aripiprazole has no appreciable affinity for cholinergic muscarinic receptors. The mechanism of action of aripiprazole, as with other drugs having efficacy in schizophrenia, is unknown. It has been proposed, however, that the efficacy of aripiprazole is mediated through a combination of partial agonist activity at D₂ and 5-HT_{1A} receptors and antagonist activity at 5-HT_{2A} receptors. Drugs effective for improving the negative symptoms and effective for improving the positive symptoms of schizophrenia are still highly desirable, more so, when the drugs can diminish the undesirable side effects.

U.S. Pat. No. 5,006,528 discloses a process for the preparation of aripiprazole with the base triethylamine and sodium iodide as catalysts in acetonitrile. Because the recovery of the triethylamine can be very difficult, the process of the application does not utilize triethylamine. The synthesis of the aripiprazole intermediate 7-(4-bromobutoxy)-3,4-dihydrocarbostyril (BBQ), using the solvent dimethylformamide, is disclosed in J. med. Chem. 1998, 41, 658-667. The recovery of DMF can also be very difficult.

Accordingly, the present invention encompasses methods of synthesizing aripiprazole, as the drug is safer than other anti-psychotic drugs such as olanzapine or ziprazidone.

### SUMMARY OF THE INVENTION

The invention encompasses processes for the preparation of aripiprazole from 7-(4-bromobutoxy)-3,4-dihydrocarbostyril (BBQ) and 1-(2,3-dichlorophenyl)piperazine hydrochloride (DCP). Typically, the preparation of aripiprazole by the methods of the invention comprises combining BBQ and DCP, in the presence of at least one base and at least one organic solvent to form a reaction mixture, heating the reaction mixture to reflux for a sufficient time to effect the reaction, and isolating aripiprazole.

Another embodiment of the invention encompasses synthesizing aripiprazole from BBQ and DCP using at least one phase transfer catalyst. Typically, the aripiprazole synthesis comprises mixing BBQ and DCP, in the presence of at least one base, and at least one phase transfer catalyst in at least one organic solvent to form a reaction mixture, heating the reaction mixture to reflux for a sufficient time to effect the reaction, and isolating aripiprazole.

### DETAILED DESCRIPTION OF THE INVENTION

The invention encompasses processes for preparing aripiprazole using the intermediate BBQ and DCP, and may optionally include the use of phase transfer catalysts.

DCP may be prepared as described in U.S. Pat. No. 5,006,528, herein incorporated by reference. BBQ may be prepared as provided in commonly assigned U.S. Application. No. [Attorney docket # 01662/78205], filed Feb. 7, 2005, hereby incorporated by reference, or as described in U.S. Pat. No. 5,006,528.

The process comprises combining BBQ and DCP in the presence of at least one base and at least one organic solvent to form a reaction mixture; heating the reaction mixture to reflux for a sufficient amount of time to effect the reaction; and isolating aripiprazole. The process of the invention is preferably performed in an inert atmosphere, such as under nitrogen gas.

DCP is present in an amount sufficient to react with BBQ, for example, in a stoichiometric amount. Typically, DCP is added in an amount of about 1 to 2 mol equivalents to BBQ. Preferably, DCP is present in an amount of about 1.2 mol equivalents.

Typically, inorganic bases are used. Typical bases include, but are not limited to, NaOH, KOH, Ca(OH)₂, Na₂CO₃, NaHCO₃, or K₂CO₃. Preferably, the base is Na₂CO₃ or K₂CO₃. The base may be present in an amount of about 1 mol equivalents to about 3 mol equivalents. Preferably, the base is present in an amount of about 1.8 to about 2.7 mol equivalents to the BBQ.

The organic solvent may be any suitable organic solvent as easily determined by one of ordinary skill in the art, such as acetonitrile, methanol, ethanol, 1-butanol, 2-butanol, or isopropanol. The solvent is preferably acetonitrile. The reaction may be maintained at reflux temperature for a time sufficient to complete the reaction. The reaction temperature is preferably maintained at about 60° C. to about 101° C. The time necessary to complete the reaction may depend on scale and mixing procedures, and may easily be determined by one skilled in the art by measuring the absence of the limiting reagent using such techniques as HPLC. Preferably, the reaction time is about 2 hours to about 24 hours, and more preferably is about 2 hours to about 20 hours. Most preferably, the reaction time is about 2 hours to about 3 hours.

The invention also encompasses the synthesis of aripiprazole using at least one phase transfer catalyst. Typically, the aripiprazole synthesis comprises mixing BBQ and 1-(2,3-dichlorophenyl)piperazine hydrochloride (DCP), in the presence of at least one base and at least one phase transfer catalyst in at least one organic solvent to form a reaction mixture; heating the reaction mixture to reflux for a sufficient amount of time to effect the reaction; and isolating aripiprazole.

The phase transfer catalyst may be present in an amount of about 0.1 to about 0.5 mol equivalents to BBQ, and is preferably present in an amount of about 0.15 to about 0.2 mol equivalents to BBQ. Several classes of compounds are known to be capable of acting as phase transfer catalysts, such as quaternary ammonium compounds and phosphonium compounds. Phase transfer catalysts include, but are not limited to, tetrabutylammonium bromide; tetrabutylammonium hydroxide; TEBA; tricaprylylmethylammonium chloride, such as Aliquat® 336 (manufactured by Aldrich Chemical Company, Inc. Milwaukee, Wis.); dodecyl sulfate, sodium salt, such as sodium lauryl sulfate; tetrabutylammonium hydrogensulfate; hexadecyl tributyl phosphonium bromide; or hexadecyltrimethylammonium bromide. Preferably, the phase transfer catalysts used in the methods of the invention include at least one of dodecyl sulfate, sodium salt, hexadecyltrimethylammonium bromide, or tetrabutylammonium bromide.

DCP is present in an amount sufficient to react with BBQ, for example, in a stoichiometric amount. Typically, DCP is added in an amount of about 1 to 2 mol equivalents to BBQ. Preferably, DCP is present in an amount of about 1.2 mol equivalents.

Typically, inorganic bases are used. Typical bases include, but are not limited to, NaOH, KOH, Ca(OH)₂, Na₂CO₃, NaHCO₃, or K₂CO₃. Preferably, the base is Na₂CO₃ or K₂CO₃. The base may be present in an amount of about 1 mol equivalents to about 3 mol equivalents. Preferably, the base is present in an amount of about 1.8 to about 2.7 mol equivalents to the BBQ.

The organic solvent may be any suitable organic solvent as easily determined by one of ordinary skill in the art, such as acetonitrile, methanol, ethanol, 1-butanol, 2□butanol, or isopropanol. The solvent is preferably acetonitrile. The reaction may be maintained at reflux temperature for a time sufficient to complete the reaction. The reaction temperature is preferably maintained at about 60° C. to about 101° C. The time necessary to complete the reaction may depend on scale and mixing procedures, and may easily be determined by one skilled in the art by measuring the absence of the limiting reagent using such techniques as HPLC. Preferably, the reaction time is about 2 hours to about 24 hours, and more preferably is about 4 hours to about 20 hours.

Isolating of aripiprazole obtained by the processes described above comprises removal of solvent; cooling of the reaction mixture; and precipitation of aripiprazole. The solvent may be removed using techniques commonly known to one skilled in the art. The solvent is preferably removed by distillation from the reaction mixture by vacuum or atmospheric pressure. The reaction mixture may then be cooled. Preferably, the reaction mixture is cooled to about 70° C.

Typically, the aripiprazole is precipitated by adding water to the cooled reaction mixture and stirring the reaction mixture. Sufficient water should be added to precipitate the product from the reaction mixture after stirring in water. Depending on the scale of the reaction and the concentration of the product, one of ordinary skill in the art can easily determine the conditions necessary to precipitate the product with little or no experimentation. Typically, in a concentrated reaction mixture, the product will precipitate after stirring the reaction mixture in water for between about 15 minutes to about 30 minutes.

Preferably, the reaction mixture is further cooled to about 40° C., and stirred overnight at this temperature. The precipitate is then preferably collected by filtration and washed with water. The washed precipitate may optionally be mixed with water again, and the mixture stirred for about half an hour. The precipitate may then be collected by filtration and washed with water a second time, yielding crude aripiprazole.

Having described the invention with reference to certain preferred embodiments, other embodiments will become apparent to one skilled in the art from consideration of the specification. The invention is further defined by reference to the following examples describing in detail the preparation of the composition of the invention. It will be apparent to those skilled in the art that many modifications, both to materials and methods, may be practiced without departing from the scope of the invention.

### EXAMPLES

### Example 1

### Preparation of Crude Aripiprazole

BBQ (10 Kg), DCP-HCl (9.85 Kg), and potassium carbonate (9.3 Kg) were mixed with acetonitrile (80 L) in a jacketed reactor equipped with a mechanical stirrer and a reflux condenser, forming a reaction mixture. The reaction mixture was heated to reflux and maintained for two hours, until the reaction was complete as determined by less than 2% of BBQ in the reaction mixture when measured by HPLC. 50 L of acetonitrile was distilled from the reaction mixture, and the reaction was cooled to 70° C. 50 L of water was added to the reaction mixture, and the reaction mixture was stirred for half an hour. The reaction mixture was cooled to 40° C. and stirred overnight at this temperature. A precipitate formed, and was collected by filtration and washed with water. 50 L of water was added to the washed precipitate, and stirred for a half an hour. The precipitate was again collected by filtration, and the precipitate washed with water. 18 Kg of wet crude aripiprazole was obtained (90% yield).

### Example 2

### Preparation of Crude Aripiprazole

BBQ (1 Kg), DCP-HCl (986 g), and potassium carbonate (927 g) were mixed with acetonitrile (6 L) in a jacketed reactor equipped with a mechanical stirrer and a reflux condenser, forming a reaction mixture. The reaction mixture was heated to reflux and maintained for three hours, until the reaction was complete as determined by less than 1% of BBQ in the reaction mixture when measured by HPLC. 3 L of acetonitrile was distilled from the reaction mixture, and the reaction was cooled to 70° C. 5 L of water was added to the reaction mixture, and the reaction mixture was stirred for half an hour. The reaction mixture was cooled to 40° C. and stirred overnight at this temperature. A precipitate formed, and was collected by filtration and washed with water. 3.5 L of water was added to the washed precipitate, and stirred for a half an hour. The precipitate is again collected by filtration and washed with water. 1.8 Kg of wet crude aripiprazole was obtained (94% yield).

### Example 3

### Preparation of Aripiprazole using Dodecyl Sulfate Sodium Salt as a Phase Transfer Catalyst

7-(4-bromobutoxy)-3,4-dihydrocarbostyril (BBQ) (4 g, 13.88 mmol, 1 eq.), 1-(2,3-dichlorophenyl)piperazine hydrochloride (DCP) (3.95 g, 17.17 mmol, 1.2 eq.), Na₂CO₃ (2.65 g, 25 mmol, 1.8 eq.), dodecyl sulfate, sodium salt (0.7 g, 2.4 mmol, 0.17 eq.) were suspended in acetonitrile (40 ml). The mixture was heated to reflux for 4 hours. The suspension volume was reduced to about one quarter of the volume, poured into 70 ml of water, and stirred for 15 minutes. A white precipitate formed was filtered and washed twice with water (50 ml). Dump crude aripiprazole (6.4 g, 90% yield) was obtained.

### Example 4

### Preparation of Aripiprazole Using Tetrabutylammonium Bromide as Catalyst

7-(4-bromobutoxy)-3,4-dihydrocarbostyril (3 g, 10.41 mmol, 1 eq.), 1-(2,3-dichlorophenyl)piperazine hydrochloride (2.96 g, 12.86 mmol, 1.2 eq.), Na₂CO₃ (2.98 g, 28 mmol, 2.7 eq), and tetrabutylammonium bromide (0.6 g, 1.86 mmol, 0.18 eq.) were suspended in acetonitrile (40 ml) and heated to reflux for 20 hours. The suspension volume was reduced to about one quarter of the original volume, poured into 70 ml of water, and stirred for 15 minutes. A white precipitate formed, was collected by filtration, and washed twice with water (50 ml). Dump crude aripiprazole was obtained (4.5 g, 70% yield).

### Example 5

### Preparation of Aripiprazole Using Hexadecyltrimethylammonium Bromide as Catalyst

7-(4-bromobutoxy) 3,4-dihydrocarbostyril (3 g, 10.41 mmol, 1 eq.), 1-(2,3-dichlorophenyl)piperazine hydrochloride (2.96 g, 12.86 mmol, 1.2 eq.), Na₂CO₃ (1.98 g, 18.7 mmol, 1.8 eq), and hexadecyltrimethylammonium bromide (0.6 g, 1.64 mmol, 0.16 eq.) were suspended in acetonitrile (40 ml) and the mixture was stirred for 20 hours at reflux. The suspension volume was reduced to about one quarter of the original volume, poured into water (70 ml), and stirred for 15 minutes. A white precipitate formed, was collected by filtration, and washed twice with water (50 ml). Dump crude aripiprazole was obtained (3.7 g, 74% yield).

### Example 6

### Preparation of Aripiprazole Using Sodium Lauryl Sulfate as a Catalyst in an Alcoholic Solvents

7-(4-bromobutoxy) 3,4-dihydroxycarbostyril (4 g, 13.9 mmol, 1 eq.), 1-(2,3-dichlorophenyl)piperazine hydrochloride (3.95 g, 17.2 mmol, 1.2 eq), Na₂CO₃ (2.65 g, 25 mmol, 1.8 eq.), and sodium lauryl sulfate (0.6 g, 2 mmol, 0.14 eq) were suspended in alcohol (40 ml) and the mixture was heated to reflux for 4 hours. The mixture was reduced to about one quarter of the volume, poured into water (70 ml), and stirred for 15 minutes. A white precipitate formed, was collected by filtration, and washed with water (2×50 ml). The product of the reaction produced with a variety of alcohol solvents is illustrated in Table 1.

TABLE 1
Aripiprazole Yields with a Variety of Solvents

| Alcohol | Aripiprazole crude amount (g) | Yield (%) |
|---|---|---|
| Ethanol | 3.9 | 74 |
| 2-Propanol | 4.3 | 64.7 |
| 1-Butanol | 3.8 | 86.5 |

1. A process for synthesizing aripiprazole comprising:
combining 7-(4-bromobutoxy)-3,4-dihydrocarbostyril and 1-(2,3-dichlorophenyl)piperazine hydrochloride, in the presence of at least one base, and at least one phase transfer catalyst in at least one organic solvent;
heating the reaction mixture for a time sufficient to effect the reaction; and
isolating the precipitate.

2. The process according to claim 1, wherein the reaction mixture is heated to a temperature of about 60° C. to about 101° C.

3. The process according to claim 1, wherein the phase transfer catalyst is at least one of tetrabutylammonium bromide; tetrabutylammonium hydroxide; TEBA; tricaprylylmethylammonium chloride; dodecyl sulfate, sodium salt; tetrabutylammonium hydrogensulfate; hexadecyl tributyl phosphonium bromide; or hexadecyl trimethyl ammonium bromide.

4. The process according to claim 1, wherein the phase transfer catalyst is present in a molar ratio of about 0.1 to 0.5 mol per mol of 7-(4-bromobutoxy)-3,4-dihydrocarbostyril.

5. The process according to claim 1, wherein the phase transfer catalyst is present in a molar ratio of about 0.15 to 0.2 mol per mol of 7-(4-bromobutoxy)-3,4-dihydrocarbostyril.

6. The process according to claim 1, wherein the 7-(4-bromobutoxy)-3,4-dihydrocarbostyril and 1-(2,3-dichlorophenyl)piperazine hydrochloride are present in a molar ratio of 1:2.

7. The process according to claim 1, wherein the base is at least one of NaOH, KOH, Ca(OH)₂, Na₂CO₃, K₂CO₃, or NaHCO₃.

8. The process according to claim 1, wherein the organic solvent is at least one of acetonitrile, methanol, ethanol, 1-butanol, 2-butanol, or isopropanol.

9. The process according to claim 8, wherein the organic solvent is acetonitrile.

10. A process for synthesizing aripiprazole comprising:
combining 7-(4-bromobutoxy)-3,4-dihydrocarbostyril and 1-(2,3-dichlorophenyl)piperazine hydrochloride, in the presence of at least one inorganic base and at least one organic solvent;
heating the reaction mixture for a time sufficient to effect the reaction; and
isolating the precipitate.

11. The process according to claim 10, wherein the reaction mixture is heated at about 60° C. to about 101° C.

12. The process according to claim 10, wherein the 7-(4-bromobutoxy)-3,4-dihydrocarbostyril and 1-(2,3-dichlorophenyl)piperazine hydrochloride are present in a molar ratio of 1:2.

13. The process according to claim 10, wherein the organic solvent is at least one of acetonitrile, methanol, ethanol, 1-butanol, 2-butanol, or isopropanol.

14. The process according to claim 10, wherein the organic solvent is acetonitrile.

15. The process according to claim 10, wherein the base is at least one of NaOH, KOH, Ca(OH)₂, Na₂CO₃, K₂CO₃, or NaHCO₃.

16. A process for synthesizing aripiprazole comprising:
combining 7-(4-bromobutoxy)-3,4-dihydrocarbostyril and 1-(2,3-dichlorophenyl)piperazine hydrochloride, in the presence of at least one base and at least one organic solvent selected form the group consisting of methanol, ethanol, 1-butanol, 2-butanol, and isopropanol;
heating the reaction mixture for a time sufficient to effect the reaction; and
isolating the precipitate.
